(19) United States
(12) Patent Application Publication
(10) Pub. No.: US 2005/0069566 A1
(43) Pub. Date: Mar. 31, 2005

(54) Foam carrier containing amphiphilic copolymeric gelling agent

(75) Inventors: Dov Tamarkin, Maccabim (IL); Doron Friedman, Karmei Yosef (IL); Meir Eini, Ness Ziona (IL)

Correspondence Address: WILMER CUTLER PICKERING HALE AND DORR LLP, 60 STATE STREET, BOSTON, MA, 02109, US

(73) Assignee: Foamix Ltd., Ness Ziona (IL)

(21) Appl. No.: 10/911,367

(22) Filed: Aug. 4, 2004

Related U.S. Application Data
(60) Provisional application No. 60/492,385, filed on Aug. 4, 2003.

(51) Int. Cl. A61K 7/00

(52) U.S. Cl. 424/401

(57) ABSTRACT

The invention relates to an alcohol-free cosmetic or pharmaceutical foam carrier including water, a hydrophobic solvent, a surface-active agent and a gelling agent. The cosmetic or pharmaceutical foam carrier does not contain aliphatic alcohols, making it non-irritating and non-drying. The alcohol-free foam carrier is suitable for inclusion of both water-soluble and oil soluble pharmaceutical and cosmetic agents.

### RELATED APPLICATIONS

The invention claims priority under 35 U.S.C. § 119(e) to co-pending U.S. application Ser. No. 60/492,385, filed Aug. 4, 2003, and entitled “Cosmetic and Pharmaceutical Foam,” which is incorporated by reference.

### FIELD OF THE INVENTION

The invention relates to an alcohol-free cosmetic or pharmaceutical foam carrier and use thereof. More specifically, the invention relates to a cosmetic or pharmaceutical foam carrier containing a hydrophobic solvent and having excellent spreading properties.

### BACKGROUND OF THE INVENTION

Foam products are used for topical applications of drugs and cosmetics. Various additives have been used to produce and stabilize foams products. Oil-in-water foamable emulsions are described that can contain fatty alcohols as a stabilizing agent and foam adjuvant. The inventors of the present invention have developed foams and foam emulsions that include foam adjuvants, namely fatty alcohols and fatty acids, as components for maintaining a stable foam formulation. See, for example, commonly assigned, co-pending application WO 2004/037225.

Hydrophobic solvents are difficult to formulate into a lather-producing or foam-producing product. Furthermore, addition of hydrophobic solvents to foamable compositions interferes with the lather forming ability of the surfactant used in the composition.

U.S. Pat. No. 5,679,324 describes an aerosol foamable fragrance composition that is translucent in its pre-dispensed state, and which forms a fast-breaking foam. The composition contains a surfactant, a propellant, a fragrance, a thickener, and a cosmetic vehicle (preferably water), wherein the ratio of the surfactant to propellant is from about 1:1 to about 1:10. Emollients are included at low levels (less than 10 wt %), as low emollient levels are needed to maintain the translucence of the composition. The resultant foam is not stable, as it apparently breaks spontaneously upon discharging from an aerosol container (with no need of any rubbing or sheer force application).

### SUMMARY OF THE INVENTION

In one aspect of the present invention, an alcohol-free foamable carrier composition containing a hydrophobic solvent is provided, which upon admixing with a liquefied gas propellant in an aerosol container releases a breakable foam that is suitable for topical administration. The alcohol-free foam carrier is suitable for inclusion of both water-soluble and oil-soluble active agents. As used herein, a foamable or foam carrier (or composition) includes formulations that are capable of forming a foam when dispensed with a suitable propellant.

The cosmetic or pharmaceutical foamable carrier according to one or more embodiments of the present invention includes water, a hydrophobic solvent, a surface-active agent and a specific gelling agent, and is free of short-chain or long-chain alcohols. Such carriers, when placed in an aerosol container and combined with a liquefied gas propellant, create a non-translucent oil-in-water emulsion that is stable in its pre-dispensed state. Liquefied gas propellant is added to the carrier in an amount of about 3-18% by weight of the total composition. Upon release from the aerosol container, the carriers form breakable foam products, which are suitable for topical or mucosal administration.

In one or more embodiments of the present invention, the foamable carrier includes a hydrophobic solvent at a concentration of 10% to about 20% by weight, or about 20% to about 75% by weight. The composition also contains about 0.1% to about 5% by weight of a surface-active agent; about 0.01% to about 5% by weight of an amphiphilic copolymeric gelling agent, and a liquefied gas propellant at a concentration of about 3% to about 18% by weight of the total composition. Water and optional ingredients are added to complete the total mass to 100% by weight. When the carrier ingredients are combined with the propellant in a container, a stable emulsion is obtained. On dispensing from an aerosol container, the foam carrier provides an expanded foam suitable for topical administration.

As used herein, all component percentages are reported as percent by weight of the total composition.

In another aspect of the present invention, the foam carrier includes non-translucent oil in water emulsion that is stable in its pre-dispensed state and is useful an alcohol-free lubricating foam. The lubricating foam includes 2-75% by weight of a hydrophobic solvent including at least 2% by weight of a silicone oil; an amount of water consisting of 25-98% by weight of the foamable carrier; a surface-active agent consisting of 0.1% to 5% by weight of the foamable carrier; a gelling agent consisting of 0.1% to 5% by weight of the foamable carrier; and a liquefied gas propellant, in an amount of about 3-18% by weight of the total composition, which, upon admixing in an aerosol container, readily facilitates release of a breakable foam, suitable for topical or mucosal administration, from the container.

In one or more embodiments, a foamable composition is provided that includes a foamable carrier as described herein and further includes at least one active agent at a therapeutically effective concentration. Such a composition is suitable for topical treatment of human and animal skin disorders or diseases. Alternatively, the composition is suitable for cosmetic treatment, for example, for cleansing, beautifying, promoting attractiveness or altering the appearance without affecting the body structure or function.

The cosmetic or pharmaceutical foamable carrier or foamable composition is flowable. The foamable carrier according to one or more embodiments of the present invention does not contain either short chain aliphatic alcohols or long chain aliphatic alcohols, making it non-irritant and non-drying. The foam carrier is suitable for inclusion of both water-soluble and oil-soluble active agents, as well as suspended active agents. In addition, cosmetic and medical disorders are identified that are best treated using the alcohol-free foam carrier and the alcohol-free cosmetic or pharmaceutical composition, and the advantages of such carrier and products are demonstrated.

The foam carrier or composition according to one or more embodiments of the present invention provides various advantages over current foam compositions.
(1) The foam is lightweight and thus, economical.
(2) The foam contains a hydrophobic solvent, in any desirable concentration, which provides a refatting and skin soothing effect.
(3) The foam contains silicone oil in a therapeutically effective concentration.
(4) The foam includes both water-soluble and oil-soluble active agents.
(5) The foam is easily spreadable, allowing treatment of large areas such as the arms, back, legs and the breast.
(6) Due to flow properties of the foam, the foam spreads effectively into folds and wrinkles, thereby providing uniform distribution and absorption of the active agent without the need of extensive rubbing.

As used herein, the term “about” when used to refer to wt. % in a composition means+10% of the reported wt. %. As used herein, the term “about” when used to refer to measured characteristics of the composition means+20% of the reported value.

### DETAILED DESCRIPTION OF THE INVENTION

The cosmetic or pharmaceutical foamable carrier according to one or more embodiments of the present invention includes water, a hydrophobic solvent, a surface-active agent and a gelling agent including an amphiphilic copolymer, and is free of short-chain or long-chain alcohols. Such compositions, when placed in an aerosol container and combined with a liquefied gas propellant, create a non-translucent oil-in-water emulsion that is stable in its pre-dispensed state. Liquefied gas propellant is added to the composition in an amount of about 3-18% by weight of the total composition. Upon release from the aerosol container, the compositions form breakable foam products, which are suitable for topical or mucosal administration.

The foam carrier or foam composition is described and can include the following components.

In one embodiment, a Class A foamable carrier composition contains about 10 to about 20% by weight hydrophobic solvent. An exemplary class A composition additionally includes about 0.1 to 5% by weight surface-active agent, about 0.1% to 5% by weight gelling agent including an amphiphilic copolymer and about 3% to 18% by weight liquefied gas propellant. Water and optional ingredients are added to complete the total mass to 100%.

In a further embodiment, a Class B foamable carrier composition contains about 20 to about 75% by weight hydrophobic solvent. An exemplary class B composition additionally includes about 0.1 to 5% by weight surface-active agent, about 0.1% to 5% by weight gelling agent including an amphiphilic copolymer, and about 3% to 18% by weight liquefied gas propellant. Water and optional ingredients are added to complete the total mass to 100%.

All % values are provided on a weight (w/w) basis.

Hydrophobic Solvent

The foam carrier or therapeutic composition includes a hydrophobic solvent. The hydrophobic solvent includes a material having solubility in distilled water at ambient temperature of less than about 1 gm per 100 mL, or less than about 0.5 gm per 100 mL, or less than about 0.1 gm per 100 mL. The hydrophobic solvent is a liquid at ambient (room) temperature, e.g., about 20-30° C.

The hydrophobic solvent content can vary between 2% to 75% by weight; however, different ranges are identified in order to facilitate a choice of an appropriate formulation according to the anticipated cosmetic or pharmaceutical need. Generally, higher hydrophobic solvent levels are more appropriate for the treatment of dry skin, and/or for the treatment of a disease that is more responsive to drugs delivered in an oily vehicle. A specific hydrophobic solvent level may be selected, for example, to facilitate regulating residence of an active agent in the skin. Another consideration in selecting a composition relates to the usability and tolerability of the product by the user. For example, in some instances, high hydrophobic solvent levels (i.e., from about 25% by weight of the composition) leave an oily feeling subsequent to application, which is not desirable in a topically applied composition. Thus, the specific hydrophobic solvent content is selected in view of the specific needs of the target population.

In one embodiment, the hydrophobic solvent is mineral oil. Mineral oil (Chemical Abstracts Service Registry number 8012-95-1) is a mixture of aliphatic, naphthalenic, and aromatic liquid hydrocarbons that are derived from petroleum. They are typically liquid; their viscosity is in the range of about 35 CST to about 100 CST (at 40° C.), and their pour point (the lowest temperature at which an oil can be handled without excessive amounts of wax crystals forming so as to prevent flow) is below 0° C. In contrast, white petrolatum, also termed “Vaseline”, is semi-solid at ambient temperature and leaves a waxy and sticky feeling after application and occasionally stains clothes. Thus, white petrolatum is not a hydrophobic solvent according to the present invention.

Yet other hydrophobic solvents include, but are not limited to, liquid oils from vegetable, marine or animal sources. Unsaturated oils are selected from the group consisting of olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oil and mixtures thereof in any proportion.

A particular class of oils includes polyunsaturated oils containing omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). By way of example, the unsaturated oil can contain at least 6% of an oil selected from omega-3 oil, omega-6 oil, and mixtures thereof.

Another class of oils is essential oils, which often exhibit a therapeutic effect. Examples of such oils are rosehip oil, which contains retinoids and is known to reduce acne and post-acne scars, and tea tree oil, which possesses antibacterial, antifungal and antiviral properties. Other examples of essential oils are basil, camphor, cardamom, carrot, citronella, clary, sage, clove, cypress, frankincense, ginger, grapefruit, hyssop, jasmine, lavender, lemon, mandarin, marjoram, myrrh, neroli, nutmeg, petitgrain, sage, tangerine, vanilla, and verbena. Other therapeutically beneficial oils are know in the art of herbal medication and are suitable for use as a hydrophobic solvent.

Another class of hydrophobic solvents includes, but is not limited to, liquid hydrophobic plant-derived oils, which are known to possess therapeutic benefits when applied topically.

A further class of hydrophobic solvents includes emollients, e.g., additives that have a soothing and moisturizing effect when applied to the skin or mucous membrane. Without derogating the generality of this term, examples of suitable emollients for use include isostearic acid derivatives, isopropyl palmitate, lanolin oil, diisopropyl dimerate, maleated soybean oil, octyl palmitate, isopropyl isostearate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, hydrogenated coco-glycerides, isononyl isononanoate, isotridecyl isononanoate, myristal myristate, triisocetyl citrate, octyl dodecanol, sucrose esters of fatty acids, octyl hydroxystearate and mixtures thereof. Other examples of other suitable emollients can also be found in the Cosmetic Bench Reference, pp. 1.19-1.22 (1996).

In a particular embodiment, the hydrophobic solvent is a mixture of mineral oil and an emollient in a ratio between about 2:8 and 8:2 on a weight basis.

A further class of hydrophobic solvents includes hydrophobic (non-water-soluble) silicon oils. Silicone oils impart skin protective properties and readily facilitate regulating residence of an active agent in the skin. The silicone oil is either a non-volatile silicone oil or a volatile silicone oil, however, water-soluble silicones, such as dimethicone copolyol are not within the scope of hydrophobic silicone oils. By way of example, the hydrophobic solvent can include at least 2% (w/w) silicone oil, at least 5% (w/w) silicone oil.

One or more hydrophobic solvents in any combination can be used.

Surface-Active Agents

The foam carrier or therapeutic composition also includes a surface-active agent. Surface-active agents (surfactants) include any agent that alters the surface properties of the oil and water components in the composition to aid in the formation of an emulsion. A surfactant's hydrophilic/lipophilic balance (HLB) describes the emulsifier's affinity toward water or oil. The HLB scale ranges from 1 (totally lipophilic) to 20 (totally hydrophilic), with 10 representing an equal balance of both characteristics. Lipophilic emulsifiers tend to form water-in-oil (w/o) emulsions; hydrophilic surfactants tend to form oil-in-water (o/w) emulsions. The HLB of a blend of two emulsifiers equals the weight fraction of emulsifier A times its HLB value plus the weight fraction of emulsifier B times its HLB value (weighted average).

Any surface-active agent, selected from anionic, cationic, non-ionic, zwitterionic, amphoteric and ampholytic surfactants, or combinations thereof may be used as surface-active agent. According to one or more embodiments of the present invention, the surface-active agent has a hydrophilic lipophilic balance (HLB) between about 9 and about 14, which is the required HLB (the HLB required to stabilize an O/W emulsion of a given oil) of most oils and hydrophobic solvents. Thus, in one or more embodiments, the composition is a single surface active agent having an HLB value between about 9 and 14, and in one or more embodiments, the foam composition contains more than one surface active agent and the weighted average of their HLB values is between about 9 and about 14.

Non-limiting examples of surfactants include polysorbates, such as polyoxyethylene (20) sorbitan monostearate (Tween 60) and polyoxyethylene (20) sorbitan monooleate (Tween 80); Polyoxyethylene (POE) fatty acid esters, such as Myrj 45, Myrj 49 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and sorbitol anhydrides, such as sorbitan monolaurate and sorbitan monolaurate-mono or diglycerides, isoceteth-20, sodium methyl cocoyl taurate, sodium methyl oleoyl taurate, sodium lauryl sulfate, triethanolamine lauryl sulfate and betaines.

In some embodiments, the surface-active agent is a non-ionic surfactant. Exemplary non-ionic surfactants include mono-, di- and tri-esters of sucrose with food fatty acids (sucrose esters), prepared from sucrose and methyl and ethyl esters of food fatty acids or by extraction from sucroglycerides. Further examples are sucrose esters with high monoester content, which have higher HLB values.

A combination of a non-ionic surfactant and an ionic surfactant (such as sodium lauryl sulphate) may be used. In one example, a non-ionic surfactant and an ionic surfactant are present in the foam carrier or composition at a ratio of between 1:1 and 20:1 or between 4:1 and 10:1.

Unlike prior art foamable compositions, low total amounts of surfactant are employed to obtain a stable foam. Surprisingly, lower surfactant levels are required to obtain a stable foamable composition, which is preferred in order to reduce skin irritations. Total surfactant level is in the range of about 0.1% to 5% by weight of the foamable composition, and can be less than 2% by weight or even less than 1% by weight.

Gelling Agents

The foam carrier or therapeutic composition also includes a gelling agent, which functions are to increases the viscosity of the aqueous phase of the emulsion, stabilize the composition and render desirable organoleptic properties to the foam.

It has been surprisingly discovered that certain gelling agents provide foam compositions that produce foams with high foam stability and an appealing organoleptic feel, even in the absence of foam stabilizing agents such as fatty acids and fatty alcohols. The gelling agent is selected from the class of amphiphilic copolymers. Amphiphilic copolymers include polymers having hydrophobic groups and hydrophilic groups or regions. These materials are referred to alternatively as “polymeric surfactants” because the hydrophilic and hydrophobic regions of the polymers serve to interact with and stabilize hydrophilic and lipophilic components, respectively, of a composition. The copolymer may be a random copolymer, a block copolymer of a graft or comb copolymer. Exemplary amphiphilic copolymers include include di-, tri- or multi-block copolymer or graft copolymer of a biodegradable polymer.

The polymeric surfactant may be an acrylate copolymer, in which hydrophobic moieties are chemically linked to hydrophilic polymer or hydrophilic moieties are attached to hydrophobic polymers to produce amphiphilic surface active and surface stabilizing agent. By way of example, suitable polymeric surfactants include cross linked copolymers of acrylic acid and a hydrophobic comonomer, such as Pemulen TR-1 and Pemulen TR-2, ETD 2020 and Carbopol 1382 (all, Acrylates/C10-30 alkyl acrylate crosspolymer), Natrosol CS Plus 330 and 430 and Polysurf 67 (all, cetyl hydroxyethyl cellulose), Aculyn 22 (acrylates/steareth-20 methacrylate copolymer), Aculyn 25 (acrylates/laureth-25 methacrylate copolymer), Aculyn 28 (acrylates/beheneth-25 methacrylate copolymer), Aculyn 46 (PEG-150/stearyl alcohol/SMDI copolymer), Stabylen 30 (acrylates/vinyl isodecanoate), Structure 2001 (acrylates/steareth-20 itaconate copolymer), Structure 3001 (acrylates/ceteth-20 itaconate copolymer) and Structure Plus (acrylates/aminoacrylates/C10-30 alkyl PEG 20 itaconate copolymer), where PEG is polyethylene glycol, PPG is polypropylene glycol.

Other exemplary amphiphilic copolymers include silicone polymers such as amphiphilic silicone polyols or copolyol, for example cetyl dimethicon copolyol and dimethicone copolyol PPG-3 oleyl ether, acetylated starch derivatives, amphiphilic modified starches, and amphiphilic block copolymers of ethylene oxide, propylene oxide and/or propylene glycol (also known as “poloxamer”).

The gelling agent may include other types of gelling agents, in combination with an amphiphilic copolymer. For example, naturally-occurring thickening agents may be included. Exemplary polymeric materials include locust bean gum, sodium alginate, sodium caseinate, egg albumin, gelatin agar, carrageenin gum sodium alginate, xanthan gum, quince seed extract, tragacanth gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), polyvinylpyrrolidone, polyvinylalcohol, guar gum, hydroxypropyl guar gum, soluble starch, cationic celluloses, cationic guars and the like and synthetic polymeric materials such as carboxyvinyl polymers, polyvinylpyrrolidone, polyvinyl alcohol polyacrylic acid polymers, polymethacrylic acid polymers, polyvinyl acetate polymers, polyvinyl chloride polymers, polyvinylidene chloride polymers and the like. Optionally, mixtures of the above compounds are contemplated. [This list is taken from earlier version. Do any of these polymers qualify as a polymeric surfactant?]

The gelling agent is present in the foam carrier or composition in an amount of about 0.1 to 5.0 wt % by weight. The gelling agent included in the foamable composition can be less than 1 wt % by weight of the foamable composition.

Propellants

The foam composition can be contained in and dispensed from a container capable of withstanding the pressure of the propellant gas and having an appropriate valve/nozzle for dispensing the composition as foam under pressure. A customary liquefied propellant can be added in the amount of about 3-18% of the total composition. Liquefied propellants are gases that exist as liquids under pressure, including hydrocarbons such as propane, isobutane and n-butane, dimethyl ether and chlorofluorocarbons (CFCs).

“Alcohol Free”

The foam carrier or foam composition is essentially free of aliphatic alcohols, unlike the composition disclosed in U.S. Pat. No. 6,126,920, which contains an aliphatic alcohol, preferably in amounts of 40-90 wt % aliphatic alcohol. Furthermore, the composition does not contain longer chain alcohols, such as fatty alcohols (long chain alcohols having 15 or more carbon atoms in their carbon chain). For the purpose of the present application, the term “alcohol free” refers to compositions that contain no more than 7.5% by weight of any aliphatic alcohol, having one to six carbon atoms in their carbon backbone, or no more than 7.5% by weight of any mixture of such aliphatic alcohols, as well as no more than 0.1% by weight fatty alcohol.

Optional Ingredients

The pharmaceutical or cosmetic foam carrier optionally includes a variety of additional ingredients, which are added in order to fine-tune the consistency of the formulation, protect the formulation components from degradation and oxidation and improve their cosmetic acceptability. Any excipient can be used, including but not limited to, stabilizing agents, antioxidants, humectants, flavoring, colorant and odorant agents and other formulation components used in the art of pharmaceutical and cosmetic formulary.

Composition and Foam Physical Characteristics

A pharmaceutical or cosmetic composition manufactured using the foam carrier according to one or more embodiments of the present invention is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foam composition or carrier includes water, hydrophobic solvents, surfactant, gelling agent and propellant, thereby creating a stable emulsion having an acceptable shelf-life of at least one year, or at least two years at ambient temperature. A feature of a product for cosmetic or medical use is long term stability. Propellants, which are a mixture of low molecular weight hydrocarbons, tend to impair the stability of emulsions. It has been observed, however, that foam compositions including amphiphilic copolymers as gelling agents are surprisingly stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam. Compositions containing semi-solid hydrophobic solvents, e.g., white petrolatum, as the main ingredients of the oil phase of the emulsion, exhibit high viscosity and poor flowability and are inappropriate candidates for a foamable composition.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administratable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

As further aspect of the foam is breakability. The breakable foam is thermally stable, yet breaks under sheer force. Sheer-force breakability of the foam is clearly advantageous over thermally-induced breakability. Thermally sensitive foams immediately collapse upon exposure to skin temperature and, therefore, cannot be applied on the hand and afterwards delivered to the afflicted area.

Another property of the foam is specific gravity, as measured upon release from the aerosol can. Typically, foams have specific gravity of less than 0.1 g/mL or less than 0.05 g/mL.

Fields of Pharmaceutical Applications

By including an appropriate therapeutic agent in the foamable carrier, the foam composition is useful in treating a patient having any one of a variety of dermatological disorders (also termed “dermatoses”), such as classified in a non-limiting exemplary manner according to the following groups:

Dermatitis including Contact Dermatitis, Atopic Dermatitis, Seborrheic Dermatitis, Nummular Dermatitis, Chronic Dermatitis of the hands and feet, Generalized Exfoliative Dermatitis, Stasis Dermatitis; Lichen Simplex Chronicus; Diaper rash;
Bacterial Infections including Cellulitis, Acute Lymphangitis, Lymphadenitis, Erysipelas, Cutaneous Abscesses, Necrotizing Subcutaneous Infections, Staphylococcal Scalded Skin Syndrome, Folliculitis, Furuncles, Hidradenitis Suppurativa, Carbuncles, Paronychial Infections, Erythrasma;
Fungal Infections including Dermatophyte Infections, Yeast Infections; Parasitic Infections including Scabies, Pediculosis, Creeping Eruption;
Viral Infections;
Disorders of Hair Follicles and Sebaceous Glands including Acne, Rosacea, Perioral Dermatitis, Hypertrichosis (Hirsutism), Alopecia, including male pattern baldness, alopecia greata, alopecia universalis and alopecia totalis; Pseudofolliculitis Barbae, Keratinous Cyst;
Scaling Papular Diseases including Psoriasis, Pityriasis Rosea, Lichen Planus, Pityriasis Rubra Pilaris;
Benign Tumors including Moles, Dysplastic Nevi, Skin Tags, Lipomas, Angiomas, Pyogenic Granuloma, Seborrheic Keratoses, Dermatofibroma, Keratoacanthoma, Keloid;
Malignant Tumors including Basal Cell Carcinoma, Squamous Cell Carcinoma, Malignant Melanoma, Paget's Disease of the Nipples, Kaposi's Sarcoma;
Reactions to Sunlight including Sunburn, Chronic Effects of Sunlight, Photosensitivity;
Bullous Diseases including Pemphigus, Bullous Pemphigoid, Dermatitis Herpetiformis, Linear Immunoglobulin A Disease;
Pigmentation Disorders including Hypopigmentation such as Vitiligo, Albinism and Postinflammatory hypopigmentation and Hyperpigmentation such as Melasma (chloasma), Drug-induced hyperpigmentation, Postinflammatory hyperpigmentation;
Disorders of Cornification including Ichthyosis, Keratosis Pilaris, Calluses and Corns, Actinic keratosis;
Pressure Sores;
Disorders of Sweating; and
Inflammatory reactions including Drug Eruptions, Toxic Epidermal Necrolysis; Erythema Multiforme, Erythema Nodosum, Granuloma Annulare.

According to one or more embodiments of the present invention, the foam composition also is useful in the therapy of non-dermatological disorders by providing transdermal delivery of an active agent that is effective against non-dermatological disorders. By way of example, such disorders include localized pain in general, as well as joint pain, muscle pain, back pain, rheumatic pain, arthritis, ostheoarthritis and acute soft tissue injuries and sports injuries. Other disorders of this class include conditions, treatable by hormone therapy, such as hormone replacement therapy, transdermal nicotine administration. The foam composition according to one or more embodiments of the present invention is also useful in the delivery of local anesthetic agents.

The same advantage is expected when the foamable composition is topically applied to mucosal membranes, the oral cavity, the vagina and the rectum.

Active Agents

The foam composition is useful and advantageous for the treatment of skin disorders and for skin care and cosmetic care. The addition of an oil having refatting, protective and moisture-retaining properties in a spreadable foam form can substitute for currently available dermatological and cosmetic creams, lotions, gels, etc.

In one or more embodiments of the present invention, the foam includes an active agent directed to the treatment of a medical disorder or a cosmetic disorder. The active agent can be categorized by the benefit it provides or by its postulated mode of action. The active agents can in some instances provide more than one benefit or operate via more than one mode of action. Therefore, classifications are made for the sake of convenience and are not intended to limit the active to that particular application or applications listed. Furthermore, foam compositions, with or without further active ingredients, are suitable for the application as “cosmeceutical” preparations.

Antibacterial Agents

One class of drugs comprises antibacterial agents. The term “antibacterial” as used herein shall include, but is not limited to, any substance being destructive to or inhibiting the growth of bacteria or any substance having the capacity to inhibit the growth of or to destroy bacteria and other microorganisms, and are used in the treatment of infectious diseases. It is well known that bacterial infections are involved in a variety of superficial disorders of the skin, eye, mucosal membrane, oral cavity, vagina and rectum. The antibacterial drug can be active against gram positive and gram-negative bacteria, protozoa, aerobic bacteria and unaerobic ones.

The antibacterial drug is selected from the group consisting of chloramphenicol, tetracyclines, synthetic and semi-synthetic penicillins, beta-lactams, quinolones, fluoroquinolnes, macrolide antibiotics, metronidazlole and metronidazole derivatives and analogs, dicarboxylic acids, such as azelaic acid, silicylates, peptide antibiotics, cyclosporines and any combination thereof at a therapeutically effective concentration. Another group of antibacterial agents is non-specific and includes strong oxidants and free radical liberating compounds, such as hydrogen peroxide, bleaching agents (e.g., sodium, calcium or magnesium hypochloride and the like) iodine, chlorohexidine and benzoyl peroxide.

Exemplary foamable compositions are particularly useful and beneficial in the prevention and treatment of secondary infections, accompanying skin-structure damage, such as in cuts, wounds, burns and ulcers. In all such cases, the present formulation is easy to use, being in foam state when applied and becoming liquid upon rubbing onto the skin.

While being useful in the prevention and treatment of infections, the antibacterial foam is also applicable for decontaminating areas, afflicted with bacterial warfare organisms, such as anthrax and smallpox.

Anti-Fungal Agents

Fungal infections are another object of treatment using the foamable composition. Superficial fungal infection of the skin is one of the commonest skin diseases seen in general practice. Dermatophytosis is probably the most common superficial fungal infection of the skin. Dermatophytosis is caused by a group of fungi capable of metabolizing the keratin of human epidermis, nails or hair. There are three genera of dermatophytes causing dermatophytosis, i.e., microsporum, trichophyton and epidermophyton.

Candidiasis is an infection caused by the yeast like fungus candida albicans or occasionally other species of candida. Clinical syndromes of candidiasis include: (a) oral candidiasis (oral thrush); (b) candidiasis of the skin and genital mucous membrane; (c) candida paronychia, which inflicts the nail; and (d) genital and vaginal candida, which inflict genitalia and the vagina.

The pharmaceutical composition can include an antifungal drug that is effective against dermatophytes and candida. The antifungal drug is selected from the group consisting of azoles, diazoles, triazoles, miconazole, fluconazole, ketoconazole, clotrimazole, itraconazole griseofulvin, ciclopirox, amorolfine, terbinafine, Amphotericin B, potassium iodide, flucytosine (5FC) and any combination thereof at a therapeutically effective concentration.

The foam composition according to one or more embodiments of the present invention is useful, for example, for the treatment of tinea corporis, tinea pedis, tinea rubrum, tinea unguium, tinea cruris, tinea barbae and tinea versicolor, as well as yeast Infections, such as candidiasis, and candidal vaginitis.

Anti-Viral Agents

Any known antiviral drugs, in a therapeutically effective concentration, can be incorporated into the foam composition. Exemplary compositions are particularly beneficial in the case of viral infections. Cold sores are caused by the herpes simplex Type 1 virus and are sometimes referred to as facial herpes. Mollusca are small viral growths that appear singly or in groups on the face, trunk, lower abdomen, pelvis, inner thighs, or penis. Shingles (herpes zoster) usually occurs only once in a lifetime, appears as a rash (clusters of blisters with a red base). Shingles is caused by the same virus responsible for chickenpox. Warts are a common, benign skin tumor caused by viral infection.

The composition can include high levels of a hydrophobic solvent for enhancing the rate of penetration and improving topical distribution of any of the above listed antiviral drugs.

Anti-Inflammatory and Antiallergic Agents

The active agent can be an anti-inflammatory or antiallergic agent. Exemplary anti-inflammatory or antiallergic agents include corticosteroids, non-steroidal anti-inflammatory drugs (NSAIDs), anti-histamines, immunosuppressants and any combination thereof at a therapeutically effective concentration.

The anti-inflammatory active agent is a corticosteroid. The corticosteroid can be selected from the group consisting of clobetasol proprionate, halobetasol proprionate, betamethasone diproprionate, betamethasone valerate, fluocinolone acetonide, halcinonide, betamethasone valerate, fluocinolone acetonide, hydrocortisone valerate, triamcinolone acetonide, hydrocortisone and any combination thereof at a therapeutically effective concentration. Since corticosteroid drugs are typically hydrophobic, suitable foam carriers include high levels of a hydrophobic solvent. The hydrophobic solvent facilitates topical distribution and enhances the rate of penetration of any of the corticosteroid drugs.

The composition may include active agents for the treatment of psoriasis. Corticosteroid ointments, greasy preparations containing little or no water, are commonly used for treating psoriasis. Their main disadvantage is in a sticky feeling subsisting for extended periods subsequent to treatment being completed thereby creating a latent inconvenience and possible discomfort to the treatment recipient. In contrast, the foam composition according to one or more embodiments of the present invention containing high levels of an oil (hydrophobic solvent) spreads very easily throughout the afflicted area and absorbs into the skin without leaving any unpleasant sensation or look. Examples of other inflammatory disorders that are treatable by a foamable composition including a steroid as an active agent are atopic dermatitis, seborrhea, seborrheic dermatitis of the face and trunk, seborrheic blepharitis, contact dermatitis, stasis dermatitis (gravitational eczema; varicose eczema), exfoliative dermatitis (erythroderma), lichen simplex chronicus, pityriasis rosea and pemphigus.

Topical antihistaminic preparations currently available include 1% and 2% diphenhydramine (Benadryl® and Caladryl®), 5% doxepin (Zonalon®) cream, phrilamine maleate, chlorpheniramine and tripelennamine, phenothiazines, promethazine hydrochloride (Phenergan®) and dimethindene maleate. These drugs, as well as additional antihistamines, can also be used.

Polyunsaturated fatty acids containing omega-3 and omega-6 fatty acids (e.g., linoleic and linolenic acid, gamma-linoleic acid (GLA), eicosapentaenoic acid (EPA) and docosahexaenoic acid (DHA) also are beneficial in the treatment of psoriasis and other skin inflammation conditions and may be included in the foamable composition.

Nonsteroidal anti-inflammatory agents (NSAIDs) are useful against skin and systemic bio-abnormalities and can be added to the foam composition. The variety of compounds encompassed by NSAIDs is well-known to those skilled in the art. Specific non-steroidal anti-inflammatory agents useful in the composition include, but are not limited to:
1) Oxicams, such as piroxicam, isoxicam, tenoxicam, sudoxicam;
2) Salicylates, such as salicylic acid, ethyl salicylate, methyl salycilate, aspirin, disalcid, benorylate, trilisate, safapryn, solprin, diflunisal, and fendosal;
3) Acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;
4) Fenamates, such as mefenamic, meclofenamic, flufenamic, niflumic, and tolfenamic acids;
5) Propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and
6) Pyrazoles, such as phenylbutazone, oxyphenbutazone, feprazone, azapropazone, and trimethazone.

Any other steroidal and nonsteroidal compounds having the capacity to prevent, alleviate the symptoms of, treat or cure inflammation processes, may be generally included as anti-inflammatory agents.

The pharmaceutical composition may include an anti-inflammatory and/or an antiallergic agent that reduces the occurrence of pro-inflammatory cytokines or inhibits the effect of pro-inflammatory cytokines.

Mixtures of any anti-inflammatory agents can be used in the composition, as well as the dermatologically acceptable salts, esters, amides, prodrugs and derivatives of these agents.

Topical application of a foam, comprising a safe and effective dose of an NSAID can be useful in the prevention and/or alleviation of the symptoms of rheumatoid arthritis, osteoarthritis and pain. Topical NSAIDs, incorporated in the foam composition can be also used in the treatment of dermatological disorders such as acne, rosacea, hair growth disorders, actinic keratosis and certain skin cancer conditions.

Local Anesthetics

The foam compositions may include an effective amount of a topical anesthetic. The topical anesthetic can be selected from the group consisting of benzocaine, lidocaine, bupivacaine, chlorprocaine, dibucaine, etidocaine, mepivacaine, tetracaine, dyclonine, hexylcaine, procaine, cocaine, ketamine, pramoxine, phenol, any pharmaceutically acceptable salts thereof and mixtures of such anesthetic agents. Any mixture of synergistically beneficial anesthetic agents is contemplated

Keratolytically Active Agents

A keratolytic agent may be included as an active agent of a foamable composition. The term “keratolytically active agent” as used herein includes a compound that loosens and removes the stratum corneum of the skin, or alters the structure of the keratin layers of skin. Keratolytically active agents are used in the treatment of dermatological disorders that involve dry skin, hyperkeratinization (such as psoriasis), skin itching (such as xerosis), acne and rosacea.

Suitable keratolytically active agents include phenol and substituted phenolic compounds. Such compounds are known to dissolve and loosen the intracellular matrix of the hyperkeratinized tissue. As such, they are used in the treatment of dermatological disorders. Dihydroxybenzene and derivatives thereof have been recognized as potent keratolytic agents. Resorcinol (m-dihydroxybenzene) and derivatives thereof are used in anti-acne preparations. In addition to hydroquinone (p-dihydroxybenzene) having anti-pigmentation properties, hydroquinone is also known to be keratolytic. These compounds also exhibit antiseptic properties. Cresols also possess bactericidal and keratolytic properties.

Vitamin A and vitamin A derivatives, also termed herein “retinoids”, such as retinoic acid, isoretinoic acid, retinol and retinal are another class of keratolytically active agents.

Another group of keratolytically active agents include alpha-hydroxy acids, such as lactic acid and glycolic acid and their respective salts and derivatives; and beta-hydroxy acids, such as salicylic acid (o-hydroxybenzoic acid) and salicylic acid salts and pharmaceutically acceptable derivatives.

Another class of keratolytically active agents includes urea and urea derivatives.

Retinoids

Another group of active agents includes retinol, retinal, all trans retinoic acid and derivatives, isomers and analogs thereof, collectively termed “retinoids”. Etretinate, actiretin, isotretinoin, adapalene and tazarotene are further examples of said retinoid isomers and analogs. Foamable compositions containing retinoids as the active drug can be used for the treatment of acne, seborrhea, various dermatoses, inflammation of the skin, mucosal membranes, vagina and the rectum, psoriasis, actinic keratosis and skin cancers, by application onto the affected area.

Insecticide and Insect Repellents Agents

Insects such as mosquitoes, biting flies, mites, gnats, fleas, chiggers, punkies, sand flies, lice and ticks can be annoying and sometimes pose a serious risk to human and animal health. In certain areas of the United States, mosquitoes can transmit diseases like equine and St. Louis encephalitis. Biting flies can inflict a painful bite that can persist for days, swell, and become infected. Ticks can transmit serious diseases like Lyme disease and Rocky Mountain spotted fever.

Insect repellents may be added to the foamable composition to protect people and animals from flying or biting insects, spiders, ticks and mites.

Examples of insect repellants include, but are not limited to, DEET (N,N-diethyl-m-toluamide), dimethyl phthalate, piperonyl butoxide and permethrin. Insect repelling terpenoids, have been reported by Hwang, et al, J. Chem. Ecol., 11, 1297 (1985); and Ruledge, J. Am. Mosquito Control Assoc. 4, 414 (1988).

A particular group of insect repellents includes the terpenoid compounds, described in U.S. Pat. No. 5,411,992, including:

(1) Terpenoid-alcohol or terpene-ols are terpenoids which have at least one hydroxyl group. Examples of terpene-ols include: C₁₀H₁₆0 compounds, perillyl alcohol, carveol, myrtenol, and cis-verbenol; C₁₀H₁₈O compounds, myrtanol, iso-pinocampheol, dihydrocarveol, isopulegol, terpineol, terpinen-4-ol, nerol, geraniol, and linalool, and C₁₀H₂₀O compounds, menthol, beta-citronellol, and dihydro-myrcenol.

(2) Terpenoid-esters are terpenoids, which have at least one ester group which is the product of the bonding of the hydroxyl group of a terpene-ol with an aliphatic carboxylic acid that can contain functional groups such as the hydroxyl or amine on the aliphatic chain. Examples of suitable aliphatic carboxylic acids include acetic acid, propionic acid, lactic acid, and various amino acids. Examples of terpenoid-esters include: carvyl acetate, carvyl propionate, and menthyl lactate.

(3) Essential oils which contain terpenoids and perfumes which contain terpenoids. Non-limiting examples of essential oils having a high content of terpene-ols and esters include bergamot (62% terpenoids); sage (>50% terpenoids); styrax (>50% terpenoids); peppermint (>50% terpenoids); and pine Siberian (75% terpenoids %). Terpenes, aldehydes and ketones vary in their usefulness but as a general group have potential as insect-repellent.

The foamable composition is particularly suitable for the effective uniform spreading of an insect repellent agent onto large areas of the skin of humans and animals. The hydrophobic solvent present in the foam composition helps retain the insect repellent on the skin surface for an extended period of time.

The foamable composition is suitable for delivery of insect-killing agents (insecticides) to an afflicted external surface area of humans and animals. Thus, the pharmaceutical or cosmetic composition includes an insecticide selected from the group consisting of permethrin, hexachlorobenzene, carbamate, naturally occurring pyrethroids, permethrin, allethrin, malathion, piperonyl butoxide and any combination thereof at a therapeutically effective concentration. The application of the composition is very convenient and it spreads easily, even over hairy areas. The hydrophobic solvent present in the foam composition helps retain the insecticide on the treated area for an extended period of time. Furthermore, the presence of a hydrophobic solvent in the foam eases mechanical removal of lice and nits with a comb.

Anti-Cancer Drugs

Anti-cancer drugs can also be used as the drug of choice for the treatment of skin malignant tumors such as basal cell carcinoma, squamous sell carcinoma, melanoma and Kaposi's sarcoma, as well as the pre-cancerous condition actinic keratosis. In certain cases, topical cytotoxic and antiproliferative drugs are used to treat or prevent such cancers, including 5-fluorouracil, also called 5-FU. 5-FU, as well as any other anti-cancer agents, know in the art of cancer medicine, can be incorporated in the foam at therapeutically effective levels. An exemplary family of anticancer drugs, suitable for usage in the foam of the present formulation comprises antiestrogens, such as tamoxifen.

Photodynamic Therapy Agents

The foam composition is also useful to deliver photo-sensitizing agents. A photosensitizer can be selected from the group consisting of poephyrins, modified porphyrins, psoralen, 8-methoxypsoralen, 5-methoxypsoralen, psoralen derivatives, chlorins, bacteriochlorins, phthalocyanines, naphthalocyanines, pheophorbides, purpurins, m-THPC, mono-L-aspartyl chlorin e6, bacteriochlorins, phthalocyanines, benzoporphyrin derivatives and photosensitizer precursors, such as aminolevulinic acid (ALA).

Active Agents for Burns, Wounds, Cuts and Ulcers

The treatment of burns, wounds, cuts and ulcers using a foamable composition is particularly advantageous. The foam can include both anti-infective agents (against bacteria, fungi and/or viruses), antiinflammatory agents (steroidal and/or NSAIDs) and pain relieving components. Upon application, the foam spreads easily, covering the surface of the affected area, and without causing pain.

Skin Care Active Agents

The foam composition is useful and advantageous for skin care and cosmetic care. The combination of oil and water having moisture-retaining properties in a spreadable foam form can be used to substitute currently used cosmetic skin care creams, lotions, gels, etc. The cosmetic foam compositions are suitable for the further application as “cosmeceutical” preparation (cosmetic products with therapeutic benefit), to treat “cosmetic” skin disorders, such as aging skin, wrinkles, hyperpigmentation (melasma, chloasma, freckles, etc.), scaly skin and other skin undesirable properties.

The CTFA Cosmetic Ingredient Handbook describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and humectants facilitating regulating the residence of an active agent in the skin), skin soothing and/or healing agents (e.g., panthenol and derivatives (e.g., ethyl panthenol), aloe vera, pantothenic acid and pantothenic acid derivatives, allantoin, bisabolol, and dipotassium glycyrrhizinate), skin treating agents, thickeners, and vitamins and derivatives thereof.

Anti-Acne Active Agents

An anti-acne agent can be included in the foamable composition. The anti-acne agent can be selected from the group consisting of resorcinol, sulfur, salicylic acid and salicylates, alpha-hydroxy acids, nonsteroidal anti-inflammatory agents, benzoyl peroxide, retinoic acid, isoretinoic acid and other retinoid compounds, adapalene, tazarotene, azelaic acid and azelaic acid derivatives, antibiotic agents, such as erythromycin and clyndamycin, zinc salts and complexes, and combinations thereof, in a therapeutically effective concentration.

Anti-Wrinkle Active Agents/Anti-Atrophy Active Agents and Agents to Treat Dry and Scaly Skin (Xerosis and Ichthyosis)

The foamable composition may also include an effective amount of an anti-wrinkle active and/or at least one anti-atrophy active. Exemplary anti-wrinkle/anti-atrophy active agents suitable for use in the foamable compositions include sulfur-containing D and L amino acids and their derivatives and salts, particularly the N-acetyl derivatives; thiols; hydroxy acids (e.g., alpha-hydroxy acids such as lactic acid and glycolic acid and their derivatives and salts; or beta-hydroxy acids such as salicylic acid and salicylic acid salts and derivatives), urea, hyaluronic acid, phytic acid, lipoic acid; lysophosphatidic acid, skin peel agents (e.g., phenol, resorcinol and the like), vitamin B3 compounds (e.g., niacinamide, nicotinic acid and nicotinic acid salts and esters, including non-vasodilating esters of nicotinic acid (such as tocopheryl nicotinate), nicotinyl amino acids, nicotinyl alcohol esters of carboxylic acids, nicotinic acid N-oxide and niacinamide N-oxide), vitamin B5 and retinoids (e.g., retinol, retinal, retinoic acid, retinyl acetate, retinyl palmitate, retinyl ascorbate). In the case of dry, scaly skin (xerosis) and ichthyosis such agents can alleviate the symptoms by temporary relief of itching associated with these conditions.

Anti-Oxidants/Radical Scavengers

An effective amount of an anti-oxidant/radical scavenger can be added to the foamable compositions, for example, in an amount from about 0.1% to about 10% (w/w), or from about 1% to about 5% (w/w).

Anti-oxidants/radical scavengers such as ascorbic acid (vitamin C) and ascorbic acid salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox.®), gallic acid and gallic acid alkyl esters, especially propyl gallate, uric acid and uric acid salts and alkyl esters, sorbic acid and sorbic acid salts, lipoic acid, amines (e.g., N,N-diethylhydroxylamine, amino-guanidine), sulfhydryl compounds (e.g., glutathione), dihydroxy fumaric acid and dihydroxy fumaric acid salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts can be used.

The foam is suitable for delivering skin protecting and revitalizing anti-oxidants/radical scavengers. Polyunsaturated fatty acids containing omega-3 and omega-6 fatty acids (e.g., linoleic and linolenic acid, gamma-linoleic acid (GLA), eicosapentaenoic acid (EPA) and docosahexaenoic acid (DHA)) are beneficial in the treatment of psoriasis and other skin inflammation conditions. Likewise, emollients and silicone oils exert moisture-retaining and skin protective effects on the skin. Thus, a skin protective foam is provided, wherein the hydrophobic solvent comprises in full or in part, a solvent, selected from the group of emollients, silicone oil and oils, rich in unsaturated fatty acids, thus, affording a synergistic therapeutic effect of the anti-oxidants/radical scavenger agent and the vehicle components.

Self-Tanning Active Agents

The foam composition is particularly suitable for the uniform delivery of a tanning active agent onto large areas of the skin. The compositions contain from about 0.1% to about 20%, or from about 2% to about 7%, or even from about 3% to about 6% of dihydroxyacetone or any other compound know in the art as an artificial tanning active agent.

Skin Lightening and Whitening Agents

The foam composition may be formulated to provide a composition for the uniform delivery of a skin lightening agent. When used, the composition contains from about 0.1% to about 10%, or from about 0.2% to about 5% of a skin-lightening agent. Suitable skin lightening or whitening agents include those known in the art, including hydroquinone, azelaic acid and other related dicarboxylic acids, and salts and derivatives thereof, retinoids, kojic acid, arbutin, nicotinic acid and nicotinic acid precursors, salts and derivatives, ascorbic acid and salts and derivatives thereof (e.g., magnesium ascorbyl phosphate or sodium ascorbyl phosphate), and herbal extracts (e.g., mulberry extract, placental extract).

In one or more embodiments of the present invention, the foam composition includes a combination of at least one skin-whitening agent and at least one additional active agent selected from retinoids, keratolytically active agents and anti-inflammatory agents.

In one or more embodiments, the composition includes a combination of at least one skin-whitening agent and at least one keratolytically active agent selected from a alpha-hydroxy acids, beta hydroxy acids, and retinoids.

In one or more embodiments of the present invention, the foam composition includes a combination of a skin-whitening agent and an inorganic sunscreen agent. When inorganic sunscreen agents, e.g. titanium dioxide and zinc oxide, are rubbed onto the skin, they leave a white coating, which provides an instant (although transient) whitening effect, which is highly desirable by the consumer, who wishes to see instant change in his/her appearance. The whitening agent, in combination with the inorganic sunscreen agent in the foam carrier can be easily and uniformly distributed on the skin surface, thereby affording an even instant whitening effect, unlike creams that are difficult to spread evenly on skin areas.

Sunscreens

Exposure to ultraviolet light can result in excessive scaling and texture changes of the stratum corneum. The foam composition may be formulated to provide a composition for the delivery of sunscreen agents by inclusion of a sunscreen active. Application of a sunscreen foam is very convenient and it spreads easily over large skin areas. The presence of a hydrophobic solvent in the foam ensures long lasting effect, even while bathing.

As used herein, “sunscreen active” includes both sunscreen agents and physical sunblocks. Suitable sunscreen actives can be organic or inorganic. Inorganic sunscreens useful herein include metallic oxides such as titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20% by weight, or from about 0.5% to about 10% by weight, or from about 1% to about 5% by weight.

A wide variety of conventional organic sunscreen actives are suitable for use herein. Specific suitable sunscreen actives include, for example, p-aminobenzoic acid, p-aminobenzoic acid salts and p-aminobenzoic acid derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and di-hydroxynaphthoic acid salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and tannic acid derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane.

An effective amount of the organic sunscreen active is used, typically from about 1% to about 20% by weight, more typically from about 2% to about 10% by weight of the composition. Exact amounts will vary depending upon the sunscreen or sunscreens chosen and the desired Sun Protection Factor (SPF). A composition containing at least one sunscreen agent having SPF of at least about 15 is useful in protecting the skin from sunburn. In one or more embodiments, a composition containing at least one sunscreen agent having SPF of at least about 15, is useful in preventing a disease comprising skin hyperpigmentation, skin cancer and other skin bioabnormalities, which are associated with excessive exposure to sun. A composition containing at least one sunscreen agent having SPF of at least about 30 can be used.

Agents for Hair Growth Disorders

Agents that affect the pattern of hair growth can be suitably incorporated in the foam composition. Male pattern baldness (MPB), the commonest cause of balding, is induced by the activity of the male hormone dihydrotestosterone (DHT), which is converted from the hormone testosterone by the enzymes 5-alpha reductase. Current treatments of MPB include minoxidil and agents, which inhibit 5-alpha reductase, such as finasteride, spironolactone, azelaic acid and azelaic acid derivatives and salts. Such agents, as well as other agents known in the art, can be incorporated in the foam composition.

Polyunsaturated fatty acids, i.e., such which include any of the essential fatty acids (EFA's), such as linoleic and linolenic acid, gamma-linoleic acid (GLA), eicosapentaenoic acid (EPA) and docosahexaenoic acid (DHA), are also known to contribute to hair growth. Thus, a hair growth foam composition is provided, in which the hydrophobic solvent comprises in full or in part, an oil, rich in such unsaturated fatty acids.

Figure-forming Agents; Agents to Treat Cellulite/Slimming

Figure forming agents such as used in the treatment of cellulite and in slimming products can be suitably incorporated in the foam composition. A non-limiting exemplary list of active agents known in the treatment of cellulite and in the induction of a slimming effect include:
Herbal extracts: baldderwack extract, butcher's, broom, cayenne, dandelion, red clover, ginkgo biloba, horse chestnut, witch hazel and borage oil
Omega 3 and omega 6 oils
Caffeic acid and salts and derivatives thereof
Xanthine agents, such as theophiline and pentoxyphilline
Nicotinic acid and salts and derivatives thereof.
   Agents to Treat Sunburn, Heat Burn, Radiation Burn, Rash and Itch

Cosmetic and pharmaceutical ingredients which are known in the art of pharmacology and cosmetology to treat dermatitis, minor skin irritations, sunburn, heat burn, radiation burn, and inhibit inflammation can be beneficially incorporated in the foam composition.

Examples of such active agents include chamomile extract (matricaria recutitia), cucumber distillate (cucumis sativus), lavender water (lavendula angustifolia), rose water (rosa damascena), witch hazel (hamamelis virginiana), allantoin, bisabolol, rosehip oil, calendula oil, azulaene, menthol and camphor.

Other Skin Care Active Agents

The active agent can be selected from the group of sulfur-containing amino acids, thiol compounds, alpha hydroxy acids, lactic acid and lactic acid derivatives and salts, glycolic acid, glycolic acid derivatives and glycolic acid salts, beta-hydroxy acids, salicylic acid and salicylic acid salts and derivatives, phytic acid, lipoic acid, lysophosphatidic acid, skin peel agents, phenol, resorcinol, vitamin B3 compounds, niacinamide, nicotinic acid and nicotinic acid salts and esters, tocopheryl nicotinate, nicotinyl amino acids, nicotinyl alcohol esters of carboxylic acids, nicotinic acid N-oxide and niacinamide N-oxide, retinoids, retinol, retinal, retinoic acid, retinyl acetate, retinyl palmitate and retinyl ascorbate, caffeine, theophilline, pentoxyphilline, dihydroxy acetone kojic acid, arbutin, nicotinic acid and nicotinic acid precursors, nicotinic acid salts, nicotinic acid derivatives, ascorbic acid, ascorbic acid salts and ascorbic acid derivatives

Use of the Foam as a Lubricating and Protective Foam

The foam, particularly the silicone oil-based foam, may be used as a lubricating foam. Typical examples are shaving foam, moisture protection foam and antifriction foam. For such purposes, the foam can be used in its the foam's basic composition (without additional formulation aids and active ingredients), or with the addition of such additives.

Further Technical Parameters

The foam composition may be placed on a patch for facilitating regulating residence of an active agent in the skin or the skin-contact compartment of a transdermal delivery apparatus and applying such object onto the skin in order to attain effective superficial treatment or enhanced penetration of the drug into the skin or through the skin. Utilizing such strategy, one can apply drugs, which are currently administered systemically or that require transdermal delivery. Examples for such drugs are nicotine, testosterone and other male hormones and male hormone precursors, estrogen and other female hormones and hormone precursors, growth hormone, insulin, caffeine, steroidal and non-steroidal antiinflammatory agents and thyroid hormone substitutes.

The general process, as typically exemplified in Example 1 can be applied in order to produce the composition of the present invention. The pharmaceutical carrier according to the present invention can also be used to prepare cosmetics for beauty purpose by adding into skin care agents and perfume.

### EXAMPLES

The invention is described with reference to the following examples. This invention is not limited to these examples and experiments, the full scope of which is set forth in the claims which follow.

### Example 1

### Production of Pharmaceutical or Cosmetic Foam Carrier and composition—General Method

The method for preparing of a pharmaceutical foam carrier generally comprised following steps.

Step 1—Aqueous Phase: Gelling agent and surface-active agent are dissolved in water, with agitation. The solution is warmed to 50-70° C. Water soluble cosmetic or pharmaceutical active Ingredients and optional water soluble ingredients are added with agitation to the Aqueous Phase mixture. In case of heat sensitive active ingredients, add the active ingredient with agitation to the mixture, after Step 3.

Step 2—Hydrophobic Phase: The hydrophobic solvent is heated to same temperature. Oil soluble cosmetic or pharmaceutical active ingredients and optional oil soluble formulation ingredients are added with agitation to the Hydrophobic Phase mixture. In case of heat sensitive active ingredients, add the active ingredient with agitation to the mixture, after Step 3.

Step 3—The warm Hydrophobic Phase is gradually poured into the warm Aqueous Phase, with agitation, followed by Ultraturax homogenization. The mixture is allowed to cool down to ambient temperature.

Step 4—The mixture, at ambient temperature, is added to an aerosol container, the container is sealed and appropriate amount of propellant (about 10% of the composition mass) is compressed into the container.

### Example 2

### Foam Compositions

This example shows that stable E-grade foams can be produced without fatty alcohol or fatty acid. The oil phase in the present example included total oil components between 10% and 36%. These oils are exchangeable with any other hydrophobic solvent, as defined hereinabove.

| | Sample No: | | | | |
|---|---|---|---|---|---|
| | 1 | 2 | 3 | 4 | 5 |
| | % w/w | % w/w | % w/w | % w/w | % w/w |
| Mineral oil | | 6.00 | 12.00 | 12.00 | 12.00 |
| Capric caprylic triglyceride | 10.00 | 6.00 | 6.00 | | 12.00 |
| Isopropyl myristate | | | | 12.00 | 12.00 |
| Polysorbate 80 | 3.00 | 3.00 | 3.00 | 3.00 | 3.00 |
| Pemulen TR1 | 0.10 | 0.10 | | | |
| Pemulen TR2 | | | 0.20 | 0.20 | 0.20 |
| Cocamidopropyl betaine | 0.50 | 0.50 | 0.30 | — | — |
| Sorbitan isostearate | 0.40 | 0.40 | 0.40 | 0.40 | 0.40 |
| Methocel K100M | 0.30 | 0.30 | — | 0.3 | 0.3 |
| Xantan Gum | — | 0.20 | — | — | — |
| Triethanolamine | 0.10 | 0.10 | 0.20 | 0.20 | 0.20 |
| EDTA disodium | — | — | — | 0.5 | 0.5 |
| Phenonip (preservative) | 0.30 | 0.30 | 0.30 | 0.30 | 0.30 |
| Propellant | 8.00 | 8.00 | 8.00 | 8.00 | 8.00 |
| Purified water | qs 100.0 | qs 100.0 | qs 100.0 | qs 100.0 | qs 100.0 |

### Example 3

### Foam Compositions with Drugs

This example demonstrates that foams with drugs can be attained having E-grade quality without fatty alcohols or fatty acids.

| | Sample No: | | | | |
|---|---|---|---|---|---|
| | 6 | 7 | 8 | 9 | 10 |
| | % w/w | % w/w | % w/w | % w/w | % w/w |
| Mineral oil | 6.00 | 6.00 | 6.00 | 12.00 | 12.00 |
| Capric caprylic triglyceride | 6.00 | 6.00 | 6.00 | | 12.00 |
| Isopropyl myristate | | | | 12.00 | 12.00 |
| Polysorbate 80 | 3.00 | 3.00 | 3.00 | 3.00 | 3.00 |
| Pemulen TR1 | 0.10 | 0.10 | 0.10 | | |
| Pemulen TR2 | | | | 0.20 | 0.20 |
| Cocamidopropyl betaine | 0.50 | 0.50 | 0.50 | — | — |
| Sorbitan isostearate | 0.40 | 0.40 | 0.40 | 0.40 | 0.40 |
| Methocel K100M | 0.30 | 0.30 | 0.30 | 0.3 | 0.3 |
| Xantan Gum | 0.20 | 0.20 | 0.20 | — | — |
| Triethanolamine | 0.10 | 0.10 | 0.10 | 0.20 | 0.20 |
| EDTA disodium | — | — | — | 0.5 | 0.5 |
| Phenonip (preservative) | 0.30 | 0.30 | 0.30 | 0.30 | 0.30 |
| Propellant | 8.00 | 8.00 | 8.00 | 8.00 | 8.00 |
| Metronidazole | 0.75 | | | | 0.75 |
| Betamethasone valerate | | 0.12 | | | |
| Miconazole | | | | 2.00 | |
| Acyclovir | | | 5.00 | | |
| Purified water | qs 100.0 | qs 100.0 | qs 100.0 | qs 100.0 | qs 100.0 |

### Example 4

### Comparative Tolerability and Acceptability Study of a Foam Composition vs. a Conventional Cream

A panel of twelve testers was requested to apply about 0.5 gr. of the foam preparation of Example 2, Sample No. 1 on one arm and 0.5 gr. of commercial cream, in a double blind fashion. They were asked to describe their feeling about the ease of application, ease of spreading, spreadability and penetrability of each of the products and to give their general rating for each of the products on a scale of 0-3 (0=poor; 1=barely acceptable; 2=acceptable and 3=excellent).

As demonstrated in the following table, the foam preparation obtained higher rates in all aspects of the test.

| | Foam | Commercial |
|---|---|---|
| | Preparation | Cream |
| Property | Mean Rating | Mean Rating |
| Ease of application | 2.3 | 2.0 |
| Ease of spreading | 2.5 | 1.8 |
| Spreadability | 2.9 | 1.5 |
| Penetrability | 2.0 | 1.7 |
| Lack of sticky feeling | 2.4 | 1.6 |
| Lack of greasy feeling | 2.2 | 1.9 |
| Lack of shiny look | 1.9 | 1.9 |
| Overall rating | 2.5 | 1.8 |

1. A non-translucent oil in water emulsion that is stable in its pre-dispensed state for use as an alcohol-free foamable carrier, comprising:
(i) about 10-75% by weight of composition of a liquid, non-volatile hydrophobic solvent;
(ii) about 0.1 to 5% by weight of a composition of a surface-active agent, having an HLB value of at least 9;
(iii) about 0.1 to 5% by weight of a gelling agent, comprising an amphiphilic copolymer; and
(iv) a liquefied gas propellant at a concentration of about 3% to about 18% by weight of the total composition.

2. The foamable carrier of claim 1, wherein said hydrophobic solvent comprises about 10-20% by weight of the composition.

3. The foamable carrier of claim 1, wherein said hydrophobic solvent comprises about 20-75% by weight of the composition.

4. The foamable carrier of claim 1, wherein said hydrophobic solvent comprises a mixture of mineral oil and an emollient in a ratio between 2:8 and 8:2 on a weight basis.

5. The foamable carrier of claim 1, wherein said surface-active agent is a mixture of a non ionic surfactant and an ionic surfactant.

6. The foamable carrier of claim 5, wherein said mixture of said non-ionic surfactant and said ionic surfactant is in a ratio of 1:1 to 20:1.

7. The foamable carrier of claim 5, wherein said mixture of said non-ionic surfactant and said ionic surfactant is in a ratio of 100:1 to 6:1.

8. The foamable carrier of claim 1, wherein said surface-active agent consists essentially of at least one non-ionic surfactant.

9. The foamable carrier of claim 1, wherein said amphiphilic copolymer is selected from the group consisting of a cross linked copolymer of acrylic acid and a hydrophobic comonomer, amphiphilic starch derivatives, amphiphilic silicon polyols or copolyols, and amphiphilic block polymers.

10. The foamable carrier of claim 1, wherein the amphiphilic copolymer is selected from the group consisting of Pemulen polymeric surfactants, acrylates/C10-30 alkyl acrylate crosspolymer, cetyl hydroxyethyl cellulose, acrylates/steareth-20 methacrylate copolymer, acrylates/laureth-25 methacrylate copolymer, acrylates/beheneth-25 methacrylate copolymer, PRG-150/stearyl alcohol/SMDI copolymer, acrylates/vinyl isodecanoate, acrylates/steareth-20 itaconate copolymer, acrylates/ceteth-20 itaconate copolymer and acrylates/aminoacrylates/C10-30 alkyl PEG 20 itaconate copolymer, amphiphilic silicone polymers, alkyl dimethicon copolyol, cetyl dimethicon copolyol, dimethicone copolyol PPG-3 oleyl ether, acetylated starch derivatives, amphiphilic modified starches, and amphiphilic block copolymers of ethylene oxide, propylene oxide and/or propylene glycol.

11. The foamable carrier of claim 9, further comprising a thickening agent selected from the group consisting of locust bean gum, sodium alginate, sodium caseinate, egg albumin, gelatin agar, carrageenin gum sodium alginate, xanthan gum, quince seed extract, tragacanth gum, starch, chemically modified starches, cellulose ethers, polyvinylpyrrolidone, polyvinylalcohol, guar gum, hydroxypropyl guar gum, soluble starch, cationic celluloses, cationic guars, carboxyvinyl polymers, polyvinyl alcohol polyacrylic acid polymers, polymethacrylic acid polymers, polyvinyl acetate polymers, polyvinyl chloride polymers, and polyvinylidene chloride polymers.

12. The foamable carrier according to claim 1, further comprising an effective amount of a therapeutically effective concentration of a drug.

13. The foamable carrier claim 1, wherein said hydrophobic solvent is selected from the group consisting of a vegetable oil, a marine oil, a mineral oil, an emollient, a silicone oil, a plant-derived therapeutic oil and any mixture thereof at any proportion.

14. The foamable carrier claim 1, wherein said surface-active agent and said gelling agent comprise less than about 8% (w/w) of the foamable composition.

15. The foamable carrier claim 1, wherein said surface-active agent and said gelling agent comprise less than about 5% (w/w) of the foamable composition.

16. A non-translucent oil in water emulsion that is stable in its pre-dispensed state, therapeutic foamable composition comprising:
(i) about 10-75% by weight of composition of a liquid, non-volatile hydrophobic solvent;
(ii) about 0.1 to 5% by weight of a composition of a surface-active agent, having HLB value of at least 9;
(iii) about 0.1 to 5% by weight of a gelling agent comprising an amphiphilic copolymer;
(iv) a therapeutically effective amount of at least one active agent; and
(v) a liquefied gas propellant at a concentration of about 3% to about 18% by weight of the total composition.

17. The therapeutic composition of claim 16, wherein said hydrophobic solvent comprises about 10-20% by weight of the composition.

18. The therapeutic composition of claim 16, wherein said hydrophobic solvent comprises about 20-75% by weight of the composition.

19. The therapeutic composition of claim 16, wherein said active agent is a drug.

20. The therapeutic composition of claim 16, wherein said active agent is a cosmetically effective agent.

21. The therapeutic composition claim 16, wherein said hydrophobic solvent is selected from the group consisting of a vegetable oil, a marine oil, a mineral oil, an emollient, a silicone oil, a plant-derived therapeutic oil and any mixture thereof at any proportion.

22. The therapeutic composition of claim 16, wherein said hydrophobic solvent includes a mixture including a mineral oil and an emollient in a ratio of substantially between 2:8 and 8:2 on a weight basis.

23. The therapeutic composition of claim 16, wherein said amphiphilic copolymer is selected from the group consisting of comprises a cross linked copolymer of acrylic acid and a hydrophobic comonomer, amphiphilic starch derivatives, amphiphilic silicon polyols or copolyols, and amphiphilic block polymers.

24. The therapeutic composition of claim 16, wherein the amphiphilic copolymer is selected from the group consisting of high molecular weight, cross linked copolymers of acrylic acid and a hydrophobic comonomer, Pemulen polymeric surfactants, acrylates/C10-30 alkyl acrylate crosspolymer, cetyl hydroxyethyl cellulose, acrylates/steareth-20 methacrylate copolymer, acrylates/laureth-25 methacrylate copolymer, acrylates/beheneth-25 methacrylate copolymer, PRG-150/stearyl alcohol/SMDI copolymer, acrylates/vinyl isodecanoate, acrylates/steareth-20 itaconate copolymer, acrylates/ceteth-20 itaconate copolymer and acrylates/aminoacrylates/C10-30 alkyl PEG 20 itaconate copolymer, amphiphilic silicone polymers, alkyl dimethicon copolyol, cetyl dimethicon copolyol, dimethicone copolyol PPG-3 oleyl ether, acetylated starch derivatives, amphiphilic modified starches, and amphiphilic block copolymers of ethylene oxide, propylene oxide and/or propylene glycol.

25. The therapeutic composition of claim 23, further comprising a thickening agent selected from the group consisting of locust bean gum, sodium alginate, sodium caseinate, egg albumin, gelatin agar, carrageenin gum sodium alginate, xanthan gum, quince seed extract, tragacanth gum, starch, chemically modified starches, cellulose ethers, polyvinylpyrrolidone, polyvinylalcohol, guar gum, hydroxypropyl guar gum, soluble starch, cationic celluloses, cationic guars, carboxyvinyl polymers, polyvinyl alcohol polyacrylic acid polymers, polymethacrylic acid polymers, polyvinyl acetate polymers, polyvinyl chloride polymers, and polyvinylidene chloride polymers.

26. The therapeutic composition of claim 16, wherein said surface-active agent is a mixture of a non-ionic surfactant and an ionic surfactant.

27. The therapeutic composition of claim 26, wherein said mixture of said non-ionic surfactant and said ionic surfactant is in a ratio of 20:1 to 1:1.

28. The therapeutic composition of claim 26, wherein said mixture of said non ionic surfactant and said ionic surfactant is in a ratio of 100:1 to 6:1.

29. The therapeutic composition of claim 16, wherein said surface-active consists essentially of at least one non-ionic surfactant.

30. The therapeutic composition of claim 16, wherein said surface-active agent and said gelling agent comprise less than about 8% (w/w) of the therapeutic composition.

31. The therapeutic composition of claim 16, wherein said surface-active agent and said gelling agent comprise less than about 5% (w/w) of the therapeutic composition.

32. The therapeutic composition of claim 19, wherein the drug is intended for the treatment of a disease, having an etiology selected from the group consisting of bacterial, fungal, viral, parasitic, inflammatory, autoimmune, allergic, hormonal, malignant and any combination thereof.

33. The therapeutic composition of claim 19, wherein said drug is selected for the treatment of a bio-abnormality.

34. The therapeutic composition of claim 19, wherein said drug is intended for the treatment of a superficial condition.

35. The therapeutic composition of claim 19, wherein said drug is selected for the treatment of a disorder of the skin, mucosal membrane, eye, ear, vagina and rectum.

36. The therapeutic composition of claim 19, wherein said drug is intended for the treatment of a disorder selected from the group consisting of dermatosis, dermatitis, bacterial Infections, fungal Infections, parasitic infections, viral infections, disorders of hair follicles and sebaceous glands, acne, rosacea, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, and non-dermatological disorders that respond to transdermal delivery of said drug.

37. The therapeutic composition of claim 19, wherein said drug is an antibacterial material.

38. The therapeutic composition of claim 37, wherein said antibacterial material is selected from the group consisting of chloramphenicol, tetracyclines, synthetic and semi-synthetic penicillins, beta-lactams, quinolones, fluoroquinolnes, macrolide antibiotics, peptide antibiotics, cyclosporines, Metronidazole, free radical generating agents, iodine, chlorohexidine, benzoyl peroxide, hydrogen peroxide and any combination thereof at a therapeutically effective concentration.

39. The therapeutic composition of claim 19, wherein said drug is an antifungal material.

40. The therapeutic composition of claim 39, wherein said antifungal drug is active against dermatophytes or candida.

41. The therapeutic composition of claim 39, wherein said antifungal drug is selected from the group consisting of azoles, diazoles, triazoles, miconazole, fluconazole, ketoconazole, clotrimazole, itraconazole griseofulvin, ciclopirox, amorolfine, terbinafine, amphotericin B, potassium iodide, flucytosine (5FC) and any combination thereof at a therapeutically effective concentration.

42. The therapeutic composition of claim 19, wherein said drug is an antiviral.

43. The therapeutic composition of claim 42, wherein said antiviral drug is selected from the group consisting of vidarabine, acyclovir, gancyclovir, nucleoside-analog reverse transcriptase inhibitors, AZT (zidovudine), ddI (didanosine), ddC (zalcitabine), d4T (stavudine), 3TC (lamivudine)], non-nucleoside reverse transcriptase inhibitors, nevirapine, delavirdine, protease Inhibitors, saquinavir, ritonavir, indinavir, nelfinavir, ribavirin, amantadine, rimantadine and interferon.

44. The therapeutic composition of claim 16, wherein said active agent is selected from the group of insecticide and insect repellant.

45. The therapeutic composition of claim 16, wherein said active agent is an antiparasite selected from the group consisting of hexachlorobenzene, carbamate, naturally occurring pyrethroids, permethrin, allethrin, malathion, piperonyl butoxide, any terpenol and derivatives thereof, and any combination thereof at a therapeutically effective concentration.

46. The therapeutic composition of claim 16, wherein said active agent is an anti-allergic agent.

47. The therapeutic composition of claim 46, wherein said antiallergic agent is selected from the group consisting of corticosteroids, non-steroidal antiinflammatory drugs, anti-histamines, immunosuppressants, immunomodulating agent and any combination thereof at a therapeutically effective concentration.

48. The therapeutic composition of claim 16, wherein said active agent is an anti-inflammatory agent.

49. The therapeutic composition of claim 48, wherein said anti-inflammatory agent is selected from the group consisting of corticosteroids, non-steroidal antiinflammatory drugs, immunosuppressants, immunomodulators and any combination thereof at a therapeutically effective concentration.

50. The therapeutic composition of claim 48, wherein said anti-inflammatory agent is selected from the group consisting of clobetasol proprionate, halobetasol proprionate, betamethasone diproprionate, betamethasone valerate, fluocinolone acetonide, halcinonide, betamethasone valerate, fluocinolone acetonide, hydrocortisone valerate, triamcinolone acetonide, hydrocortisone and any combination thereof at a therapeutically effective concentration.

51. The therapeutic composition of claim 48, wherein said anti-inflammatory agent is a nonsteroidal anti-inflammatory drug.

52. The therapeutic composition of claim 48, wherein said anti-inflammatory agent is selected from the group consisting of oxicams, piroxicam, isoxicam, tenoxicam, sudoxicam, salicylates, aspirin, disalcid, benorylate, trilisate, safapryn, solprin, diflunisal, fendosal, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofenic, pyrazoles, phenylbutazone, oxyphenbutazone, feprazone, azapropazone and trimethazone.

53. The therapeutic composition of claims 48, wherein said anti-inflammatory agent reduces the occurrence of pro-inflammatory cytokines or inhibits the effect of pro-inflammatory cytokines.

54. The therapeutic composition of claim 46, wherein said antiallergic agent is selected from the group consisting of diphenhydramine, doxepin, phrilamine maleate, chlorpheniramine and tripelennamine, phenothiazines, promethazine hydrochloride, dimethindene maleate and any combination thereof at any therapeutically effective concentration.

55. The therapeutic composition of claim 16, wherein said active agent is an anticancer agent.

56. The therapeutic composition of claim 16, wherein said active agent is a photodynamic therapy agent.

57. The therapeutic composition of claim 19, wherein said drug is a local anesthetic agent.

58. The therapeutic composition of claim 54, wherein said anesthetic is selected from the group consisting of benzocaine, lidocaine, bupivacaine, chlorprocaine, dibucaine, etidocaine, mepivacaine, tetracaine, dyclonine, hexylcaine, procaine, cocaine, ketamine, pramoxine, phenol

59. The therapeutic composition of claim 19, wherein said drug is s nonsteroidal anti-inflammatory drug (NSAID).

60. The therapeutic composition of claim 16, wherein said active agent is a retinoid.

61. The therapeutic composition of claim 60, wherein said retinoid is selected from the group consisting of retinol, retinal, retinoic acid, etretinate, actiretin, isotretinoin, adapalene and tazarotene.

62. The therapeutic composition of claim 16, wherein said active agent is an anti-wrinkle agent.

63. The therapeutic composition of claim 16, wherein said active agent is selected from the group consisting of sulfur-containing amino acids, thiol compounds, alpha hydroxy acids, lactic acid and lactic acid derivatives and salts, glycolic acid, glycolic acid derivatives and glycolic acid salts, beta-hydroxy acids, salicylic acid and salicylic acid salts and derivatives, phytic acid, lipoic acid, lysophosphatidic acid, skin peel agents, phenol, resorcinol, vitamin B3 compounds, niacinamide, nicotinic acid and nicotinic acid salts and esters, tocopheryl nicotinate, nicotinyl amino acids, nicotinyl alcohol esters of carboxylic acids, nicotinic acid N-oxide and niacinamide N-oxide, retinoids, retinol, retinal, retinoic acid, retinyl acetate, retinyl palmitate and retinyl ascorbate, caffeine, theophilline, pentoxyphilline, dihydroxy acetone kojic acid, arbutin, nicotinic acid and nicotinic acid precursors, nicotinic acid salts, nicotinic acid derivatives, ascorbic acid, ascorbic acid salts and ascorbic acid derivatives.

64. The therapeutic composition of claim 16, wherein said active agent is a radical scavenger.

65. The therapeutic composition of claim 16, wherein said active agent is a herbal extract.

66. The therapeutic composition of claim 16, wherein said active agent is selected from the group consisting of, ascorbyl esters of fatty acids, magnesium ascorbyl phosphate, sodium ascorbyl phosphate, ascorbyl sorbate, tocopherol, tocopherol sorbate, tocopherol acetate, other esters of tocopherol, butylated hydroxy benzoic acids and their salts, 6-hydroxy-2,5,7,8-tetramethylchroman-2-carboxylic acid, gallic acid and gallic acid alkyl esters, propyl gallate, uric acid, uric acid salts and alkyl esters, sorbic acid and sorbic acid salts, lipoic acid, N,N-diethylhydroxylamine, amino-guanidine, sulfhydryl compounds, glutathione, dihydroxy fumaric acid and fumaric acid salts, lycine pidolate, arginine pilolate, nordihydroguaiaretic acid, bioflavonoids, curcumin, lysine, methionine, proline, superoxide dismutase, silymarin, tea extract, grape skin/seed extract, melanin, and rosemary extract.

67. The therapeutic composition of claim 16, wherein said active agent is a self-tanning agent.

68. The therapeutic composition of claim 16, wherein said active agent is an anti-acne active agent.

69. The therapeutic composition of claim 16, wherein said active agent is selected from the group consisting of resorcinol, sulfur, salicylic acid, salicylate salts, benzoyl peroxide, retinoic acid, isotretinoin, adapalene, tazarotene, azelaic acid and azelaic acid derivatives, antibiotic agents, erythromycin and clyndamycin and zinc salts and complexes.

70. The therapeutic composition of claim 16, wherein said active agent is a skin whitening agents.

71. The therapeutic composition of claim 68, further comprising at least one agent, selected from the group consisting of: a retinoid, a keratolytically active agent and an anti-inflammatory agent.

72. The therapeutic composition of claim 16, further comprising a sunscreen agent.

73. The therapeutic composition of claim 72, wherein said sunscreen agent is selected from the group consisting of: a UVA absorber and a UVB absorber.

74. The therapeutic composition of claim 19, wherein said drug is selected for transdermal delivery.

75. The foamable carrier of claim 16, further comprising a decontaminating agent selected from the group consisting of an oxidizing agent, iodine, iodine compounds, chlorohexidine, bleaching agent and surface-active agent.

76. A method of treating, alleviating or preventing a dermatological disorder, comprising topically administering to an afflicted area a therapeutically effective amount of a non-translucent oil in water emulsion, stable in its pre-dispensed state, breakable therapeutic foam composition comprising:
(i) about 10-75% by weight of composition of a liquid, non-volatile hydrophobic solvent;
(ii) about 0.1 to 5% by weight of a composition of a surface-active agent having HLB value of at least 9;
(iii) about 0.1 to 5% by weight of a gelling agent comprising an amphiphilic copolymer;
(iv) a therapeutically effective amount of at least one active agent; and
(v) a liquefied gas propellant at a concentration of about 3% to about 18% by weight of the total composition

77. The method according to claim 76, wherein said hydrophobic solvent includes a mixture of a mineral oil and an emollient in a ratio between 2:8 and 8:2 on a weight basis.

78. The method according to claim 76, wherein said surface-active agent is a mixture of a non ionic surfactant and an ionic surfactant in a 1:1 to 20:1 ratio.

79. The method according to claim 76, wherein said surface-active agent is substantially non ionic.

80. The method according to claim 76, wherein said amphiphilic copolymer is selected from the group consisting of a cross linked copolymer of acrylic acid and a hydrophobic comonomer, amphiphilic starch derivative, amphiphilic silicon polyols or copolyols, and amphiphilic block polymers.

81. The method according to claim 76, wherein the amphiphilic copolymer is selected from the group consisting of cross linked copolymers of acrylic acid and a hydrophobic comonomer, Pemulen polymeric surfactants, Acrylates/C10-30 alkyl acrylate crosspolymer, cetyl hydroxyethyl cellulose, acrylates/steareth-20 methacrylate copolymer, acrylates/laureth-25 methacrylate copolymer, acrylates/beheneth-25 methacrylate copolymer, PRG-150/stearyl alcohol/SMDI copolymer, acrylates/vinyl isodecanoate, acrylates/steareth-20 itaconate copolymer, acrylates/ceteth-20 itaconate copolymer and acrylates/aminoacrylates/C10-30 alkyl PEG 20 itaconate copolymer, amphiphilic silicone polymers, alkyl dimethicon copolyol, cetyl dimethicon copolyol, dimethicone copolyol PPG-3 oleyl ether, acetylated starch derivatives, amphiphilic modified starches; and amphiphilic block copolymers of ethylene oxide, propylene oxide and/or propylene glycol.

82. The method according to claim 80, further comprising a thickening agent selected from the group consisting of locust bean gum, sodium alginate, sodium caseinate, egg albumin, gelatin agar, carrageenin gum sodium alginate, xanthan gum, quince seed extract, tragacanth gum, starch, chemically modified starches, cellulose ethers, polyvinylpyrrolidone, polyvinylalcohol, guar gum, hydroxypropyl guar gum, soluble starch, cationic celluloses, cationic guars, carboxyvinyl polymers, polyvinyl alcohol polyacrylic acid polymers, polymethacrylic acid polymers, polyvinyl acetate polymers, polyvinyl chloride polymers, and polyvinylidene chloride polymers.

83. The method according to claim 76, wherein said non-ionic surfactant comprises a sucrose ester.

84. The method according to claim 76, wherein said active agent is a drug selected for the treatment of a disease, the etiology of which is selected from the group consisting of: bacterial, fungal, viral, parasitic, inflammatory, autoimmune, allergic, hormonal, malignant and combinations thereof.

85. The method according to claim 76, wherein said drug is selected from the group consisting of an antibacterial, an antifungal, an anti-inflammatory, an antiallergic drug, nonsteroidal anti-inflammatory, retinoid, alpha hydroxy acid, beta hydroxy acid, keratolytic, antiproliferative, anticancer and anti-pigmentation drugs.

86. The method according to claim 76, wherein said active agent is selected from the group consisting of an insecticide and an insect repellent.

87. The method of claim 76, wherein said active agent is selected for treating a dermatological disorder selected from the group consisting of dermatosis, dermatitis, bacterial Infections, fungal Infections, parasitic infections, viral infections, disorders of hair follicles and sebaceous glands, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, and non-dermatological disorders, which respond to transdermal delivery of said active agent.

88. The method of claim 76, wherein said active agent is a hair growth enhancer.

89. The method of claim 76, wherein said active agent substantially limits or prevents hair growth.

90. The method of claim 76, wherein said active agent is an exfoliant.

91. The method of claim 76, wherein said active agent is an epilating agent.

92. The method of claim 76, wherein said active agent is a depilating agent.

93. The method of claim 76, further comprising a sunscreen agent and a skin whitening agent.

94. A method of preventing skin cancer or preventing skin hyperpigmentation, comprising:
topically administering to a subject in risk a therapeutically effective amount of a non-translucent oil in water emulsion, stable in its pre-dispensed state, composition comprising:
(i) about 10-75% by weight of composition of a liquid, non-volatile hydrophobic solvent;
(ii) about 0.1 to 5% by weight of a composition of a surface-active agent;
(iii) about 0.1 to 5% by weight of a gelling agent comprising an amphiphilic copolymer;
(iv) a liquefied gas propellant at a concentration of about 3% to about 18% by weight of the total composition; and
(v) at least one sunscreen agent, providing SFP value of at least about 30.

95. The therapeutic composition of claims 46, wherein said antiallergic agent reduces the occurrence of pro-inflammatory cytokines or inhibits the effect of pro-inflammatory cytokines.
